(19) United States
(12) Patent Application Publication
(10) Pub. No.: US 2025/0378556 A1
(43) Pub. Date: Dec. 11, 2025

(54) ENDOSCOPIC EXAMINATION ASSISTANCE DEVICE, ENDOSCOPIC EXAMINATION SYSTEM, PROCESSING METHOD, AND STORAGE MEDIUM

(71) Applicants: NEC Corporation, Tokyo (JP); NATIONAL CANCER CENTER, Tokyo (JP)

(72) Inventors: Kenichi KAMIJO, Tokyo (JP); Tatsu KIMURA, Tokyo (JP); Yutaka SAITO, Tokyo (JP); Hiroyuki TAKAMARU, Tokyo (JP)

(73) Assignees: NEC Corporation, Tokyo (JP); NATIONAL CANCER CENTER, Tokyo (JP)

(21) Appl. No.: 19/228,890

(22) Filed: Jun. 5, 2025

(30) Foreign Application Priority Data
Jun. 7, 2024 (JP) 2024-093024

(51) Int. Cl. G06T 7/00; A61B 1/00; G06T 7/60

(52) U.S. Cl. CPC G06T 7/0012; A61B 1/00009; A61B 1/0002; A61B 1/00055; G06T 7/60; G06T 2207/10068; G06T 2207/20081; G06T 2207/30096

(57) ABSTRACT

The endoscopic examination assistance device detects at least one lesion contained in an endoscopic image obtained during an endoscopic examination; estimates an infiltration state of the lesion detected from the endoscopic image; determines whether the infiltration state exceeds a predetermined threshold value; and displays the infiltration state relative to the threshold value in a visually discernible manner. The endoscopic examination assistance device can assist user's decision making for diagnosing lesions.

### CROSS-REFERENCE TO RELATED APPLICATIONS

This application is based upon and claims the benefit of priority from Japanese patent application No. 2024-093024, filed on Jun. 7, 2024, the disclosure of which is incorporated herein in its entirety by reference.

### TECHNICAL FIELD

The present disclosure relates to an endoscopic examination assistance device, an endoscopic examination system, a processing method, and a storage medium.

### BACKGROUND ART

An endoscope may be used during the examination of internal organs. Japanese Unexamined Patent Application, First Publication No. 2011-255006 (Patent Document 1), as related art, discloses a technique for an image processing device that prevents alert images from obstructing diagnosis, treatment, or similar procedures.

### SUMMARY

In the technique related to the image processing device disclosed in Patent Document 1, there is a need for techniques that can assist examiners, including doctors.

Each example aspect of the present disclosure is intended to provide an endoscopic examination assistance device, an endoscopic examination system, a processing method, and a program capable of solving the problems mentioned above.

According to an example aspect of the present disclosure, an endoscopic examination assistance device includes: a lesion detection means that detects at least one lesion contained in an endoscopic image obtained during an endoscopic examination; an infiltration determination means that estimates an infiltration state of the lesion detected from the endoscopic image and determines whether the infiltration state exceeds a predetermined threshold value; and a display control means that displays the infiltration state relative to the threshold value in a visually discernible manner.

According to another example aspect of the present disclosure, an endoscopic examination system includes: the endoscopic examination assistance device; and a display device that displays a screen under the control of the endoscopic examination assistance device.

According to another example aspect of the present disclosure, a processing method includes: detecting at least one lesion contained in an endoscopic image obtained during an endoscopic examination; estimating an infiltration state of the lesion detected from the endoscopic image and determining whether the infiltration state exceeds a predetermined threshold value; and displaying the infiltration state relative to the threshold value in a visually discernible manner.

According to another example aspect of the present disclosure, a non-transitory storage medium storing a program causes a computer to execute steps of: detecting at least one lesion contained in an endoscopic image obtained during an endoscopic examination; estimating an infiltration state of the lesion detected from the endoscopic image and determining whether the infiltration state exceeds a predetermined threshold value; and displaying the infiltration state relative to the threshold value in a visually discernible manner.

### BRIEF DESCRIPTION OF THE DRAWINGS

FIG. 1 is a diagram showing a configuration example of an endoscopic examination system according to some example embodiments of the present disclosure.

FIG. 2 is a diagram showing a configuration example of an image processing device according to some example embodiments of the present disclosure.

FIG. 3 is a first diagram for describing a display processing flow based on the infiltration distance of a lesion site, performed by the image processing device according to some example embodiments of the present disclosure.

FIG. 4 is a second diagram showing a display processing flow based on the infiltration distance of a lesion site, performed by the image processing device according to some example embodiments of the present disclosure.

FIG. 5 is a diagram showing an example of the functional blocks of the image processing device according to some example embodiments of the present disclosure.

FIG. 6 is a diagram for describing the processing performed by an infiltration distance estimation unit according to some example embodiments of the present disclosure.

FIG. 7 is a first diagram for describing an alert generated by a display control unit according to some example embodiments of the present disclosure.

FIG. 8 is a second diagram for describing an alert generated by the display control unit according to some example embodiments of the present disclosure.

FIG. 9 is a third diagram for describing an alert generated by the display control unit according to some example embodiments of the present disclosure.

FIG. 10 is a diagram showing a processing flow example of the endoscopic examination system according to some example embodiments of the present disclosure.

FIG. 11 is a schematic configuration diagram of the endoscopic examination system according to some example embodiments of the present disclosure.

FIG. 12 is a diagram showing a configuration example of the image processing device according to some example embodiments of the present disclosure.

FIG. 13 is a diagram showing a processing flow example of the image processing device according to some example embodiments of the present disclosure.

### EXAMPLE EMBODIMENT

Hereinafter, preferred example embodiments will be described in detail, with reference to the drawings.

### First Example Embodiment

### (Configuration of Endoscopic Examination System)

An endoscopic examination system 100 according to an example embodiment of the present disclosure will be described, with reference to the drawings. The endoscopic examination system 100 is a system capable of assisting minimally invasive medical procedures by optimizing endoscopic resection of a lesion site through presenting information regarding the infiltration distance of a region of a subject suspected of being a lesion (hereinafter, may be referred to as “lesion site”) to an examiner such as a doctor who conducts an examination using an endoscope. Examples of lesions include tumors, among others. However, lesions are not limited to tumors and may include other types of lesions.

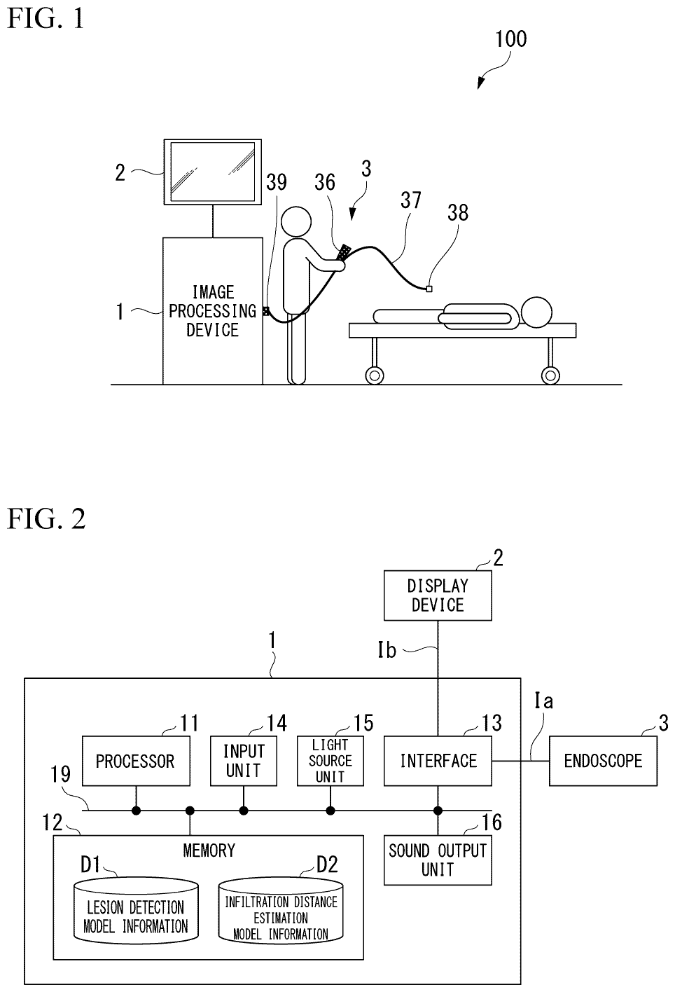

FIG. 1 is a diagram showing a configuration example of the endoscopic examination system 100 according to some example embodiments of the present disclosure. As shown in FIG. 1, the endoscopic examination system 100 includes an image processing device 1 (an example of an endoscopic examination assistance device), a display device 2, and an endoscope 3.

The image processing device 1 acquires from the endoscope 3, images (hereinafter, may be referred to as “endoscopic images Ia”) captured by the endoscope 3 in time series, and causes the display device 2 to display a screen based on the endoscopic images Ia. The endoscopic image Ia is an image captured at a predetermined frame period during at least either the insertion process or ejection process of the endoscope 3 into or from a subject such as a patient. In the present example embodiment, in a case where the image processing device 1 detects an endoscopic image Ia containing a lesion site (hereinafter, may be referred to as “lesion containing image”), it estimates the infiltration distance of the lesion (that is, the depth of the lesion) in the subject's area within the lesion-containing image. Then, the image processing device 1 causes the display device 2 to display an image based on the estimated infiltration distance. An example of the “image based on infiltration distance”, as will be described later, is an image where the image processing device 1 displays the infiltration state relative to a predetermined threshold value on the display device 2 in a visually discernible manner. Examples of the predetermined threshold value include a threshold value (for example, 1000 micrometers) that serves as a criterion for determining the applicability of endoscopic resection to a lesion site. The image processing device 1 (an example of an endoscopic examination assistance device) can be used to assist the decision-making process of a user (for example, an examiner such as doctor) who diagnoses a lesion.

The display device 2 is a display or similar device that provides a predetermined display based on a display signal supplied from the image processing device 1. The display signal is, for example, a signal used to display an image in a visually discernible manner that indicates the infiltration state relative to a predetermined threshold.

As shown in FIG. 1, the endoscope 3 includes an operation unit 36, a shaft 37, a distal end unit 38, and a connection unit 39. The operation unit 36 accepts input from an examiner for performing a predetermined operation. The operation unit 36 includes a button (hereinafter, may be referred to as a “still-image save button”) that instructs to capture (that is, save as a still image) the endoscopic image displayed on the display device 2 in the case where the examiner determines that an endoscopic image containing a lesion site is displayed on the display device 2. The shaft 37 is flexible and is inserted into an internal organ of a subject to be imaged. The distal end unit 38 incorporates an imaging unit such as a micro-imaging element. The connection unit 39 connects the endoscope 3 to the image processing device 1.

The configuration of the endoscopic examination system 100 shown in FIG. 1 is only an example, and may be modified in various ways. For example, the image processing device 1 may be integrated with the display device 2. In another example, the image processing device 1 may be composed of multiple devices.

Moreover, examples of subjects for endoscopic examination in the present disclosure include the large intestine. However, the subject of the endoscopic examination in the present disclosure is not limited to the large intestine but may be any organ amenable to endoscopic examination, such as the esophagus, stomach, and pancreas. Furthermore, examples of endoscopes applicable in the present disclosure include, but are not limited to, a pharyngoscope, bronchoscope, upper gastrointestinal endoscope, duodenal endoscope, small intestinal endoscope, colonoscope, capsule endoscope, thoracoscope, laparoscope, cystoscope, cholangioscope, arthroscope, spinal endoscope, intravascular endoscope, and epiduroscope.

### (Configuration of Image Processing Device)

FIG. 2 is a diagram showing a configuration example of the image processing device 1 according to some example embodiments of the present disclosure. It should be noted that the display device 2 and the endoscope 3 are also shown in FIG. 2. As shown in FIG. 2, the image processing device 1 includes a processor 11, a memory 12, an interface 13, an input unit 14, a light source unit 15, and a sound output unit 16. The processor 11, the memory 12, the interface 13, the input unit 14, the light source unit 15, and the sound output unit 16 are connected to one another via a data bus 19, as shown in FIG. 2.

The processor 11 performs predetermined processing by executing a program and other data stored in the memory 12. The processor 11 is a processor such as a Central Processing Unit (CPU), a Graphics Processing Unit (GPU), or a Tensor Processing Unit (TPU). The processor 11 may be composed of multiple processors. The processor 11 is an example of a computer.

The memory 12 is composed of a volatile memory such as a Random Access Memory (RAM) and a non-volatile memory such as a Read Only Memory (ROM). The volatile memory is used as a working memory. The non-volatile memory stores various types of information required for the processing of the image processing device 1. It should be noted that the memory 12 may include an external storage device either connected to or integrated into the image processing device 1. Examples of the external storage device include a hard disk. The memory 12 may also include a storage medium. Examples of the storage medium include a removable flash memory. The memory 12 stores a program for the image processing device 1 to execute various processes in the present example embodiment.

The memory 12 also stores lesion detection model information D1 and infiltration distance estimation model information D2. The lesion detection model information D1 is information relating to a lesion detection model that is a model for detecting an endoscopic image Ia that becomes a lesion-containing image from an input endoscopic image Ia. The infiltration distance estimation model information D2 is information relating to an infiltration distance estimation model that is a model for estimating the infiltration distance of a lesion site contained in an input image. The lesion detection model information D1 and the infiltration distance estimation model information D2 will be described in detail later.

The interface 13 enables the transmission and reception of information and light between the image processing device 1 and an external device. For instance, the interface 13 supplies display information “Ib”, generated by the processor 11, to the display device 2. The interface 13 also supplies light, generated by the light source unit 15, to the endoscope 3. Moreover, the interface 13 supplies to the processor 11 an electrical signal representing the endoscopic image Ia, which is provided from the endoscope 3. The interface 13 may be a communication interface such as a network adapter for performing wired or wireless communication with an external device. Furthermore, the interface 13 may be a hardware interface compliant with standards such as Universal Serial Bus (USB) and Serial AT Attachment (SATA).

The input unit 14 generates an input signal based on an operation performed by an examiner. Examples of the input unit 14 include buttons, a touch panel laminated on the display device 2, a remote controller, and a voice input device. The light source unit 15 generates light to be supplied to the distal end unit 38 of the endoscope 3. It should be noted that the light source unit 15 may also incorporate a pump or similar mechanism for sending out water and air to the endoscope 3. The sound output unit 16 outputs sound based on the control of the processor 11.

Next, the lesion detection model information D1 and the infiltration distance estimation model information D2 stored in the memory 12 will be described in detail.

The lesion detection model information D1 is information relating to a lesion detection model that, upon receiving an endoscopic image Ia as input, outputs information indicating whether or not the input endoscopic image Ia contains a lesion site. The lesion detection model information D1 includes parameters required to configure the lesion detection model. The lesion detection model is, for example, a classification model that, upon receiving an input of an endoscope image Ia, outputs a classification result regarding the presence or absence of a lesion site in the input endoscopic image Ia. The lesion detection model may be implemented using any machine learning model, including statistical models, such as neural networks or support vector machines. Representative examples of such neural network models include Fully Convolutional Network, SegNet, U-Net, V-Net, Feature Pyramid Network, Mask R-CNN, and DeepLab. In the case where the lesion detection model is implemented using a neural network, the lesion detection model information D1 includes various parameters, such as the layer architecture, neuron configuration of each layer, number and size of filters per layer, and weights of individual elements within each filter, for example.

The infiltration distance estimation model information D2 is information relating to an infiltration distance estimation model that is a model for estimating the infiltration distance of the lesion site in the image in a case where an image capturing a region of the subject containing the lesion site is input. The infiltration distance estimation model information D2 includes parameters required to configure the infiltration distance estimation model. The infiltration distance estimation model is a model that has learned the relationship between the image input to the infiltration distance estimation model and the infiltration distance of the lesion site of the subject represented in the image. The infiltration distance estimation model may be implemented using any machine learning model, including statistical models, such as neural networks or support vector machines, for example. For example, in the case where the infiltration distance estimation model is implemented using a neural network, the infiltration distance estimation model information D2 includes various parameters, such as the layer architecture, neuron configuration of each layer, number and size of filters per layer, and weights of individual elements within each filter. The infiltration distance estimation model information D2 may find the infiltration distance by adopting the infiltration distance of the central part of the lesion image as the maximum value in a case where estimating the infiltration distance. Moreover, the infiltration distance estimation model information D2 may find the infiltration distance by adopting the maximum infiltration distance in the entire image containing the lesion as the maximum value in a case where estimating the infiltration distance.

As will be described later, the image input to the infiltration distance estimation model may be a partial image of a lesion-containing image that is regularly cut out (for example, in a grid pattern) from the lesion containing image. Moreover, the image input to the infiltration distance estimation model may be the lesion-containing image itself. For example, in the case where the image input to the infiltration distance estimation model is a partial image of a lesion containing image that is regularly cut out from the lesion-containing image, the infiltration distance estimation model outputs a numerical value indicating the estimated infiltration distance at the center position of the input partial image. Furthermore, in the case where the image input to the infiltration distance estimation model is a lesion-containing image itself, the infiltration distance estimation model outputs an image showing the estimated infiltration distance for each pixel of the entire input lesion containing image (or it may be in blocks of multiple pixels or in sub-pixel units).

Moreover, the infiltration distance estimation model may be a model that outputs, in addition to the infiltration distance, an estimation result regarding the depth of each layer that constitutes the wall layer of the subject shown in the image input to the infiltration distance estimation model. For example, in the case where the subject is the large intestine, the infiltration distance estimation model may be configured to estimate the depth of each layer, including the mucosal layer, the muscularis mucosae, the submucosa, the muscularis propria, the subserosa, and the serosa. Also, in the case where the subject is the esophagus, the infiltration distance estimation model may be configured to estimate the depth of each layer, including the mucosal layer, the submucosa, the muscularis propria, and the adventitia. It should be noted that the model configured to estimate the depth of each layer forming the wall layer may be a model separate from the infiltration distance estimation model.

Moreover, in the case where the lesion detection model and the infiltration distance estimation model are learning models, each of these models may be trained preliminarily based on pairs consisting of input images conforming to the input format of each model and ground truth data representing the expected ground truth output in a case where such input images are provided to the respective models. For example, the input images used for training the lesion detection model may be endoscopic images, and the ground truth data may be pathological images (that is, images indicating lesions). Moreover, for example, the input image used for training the infiltration distance estimation model may be images of lesion sites captured in endoscopic images, and the ground truth data may be information indicating the infiltration distance of the lesion. Then, the parameters and related data of each model obtained through training may be stored in the memory 12 as the lesion detection model information D1 and the infiltration distance estimation model information D2, respectively.

### (Display Processing Performed by Image Processing Device)

The display processing performed by the image processing device 1 based on the infiltration distance of the lesion site will now be described.

### (Overview of Display Processing)

In a case where the image processing device 1 detects an endoscopic image Ia that is a lesion-containing image, the image processing device 1 estimates the infiltration distance at each position of the subject shown in the lesion-containing image. Then, the image processing device 1 causes the display device 2 to display the infiltration state relative to a predetermined threshold value in a visually discernible manner based on the estimated infiltration distance. As a result, by presenting information regarding the infiltration distance of the lesion site to the examiner, such as doctor conducting an examination using an endoscope, the image processing device 1 can optimize endoscopic resection of the lesion site and facilitate minimally invasive medical procedures.

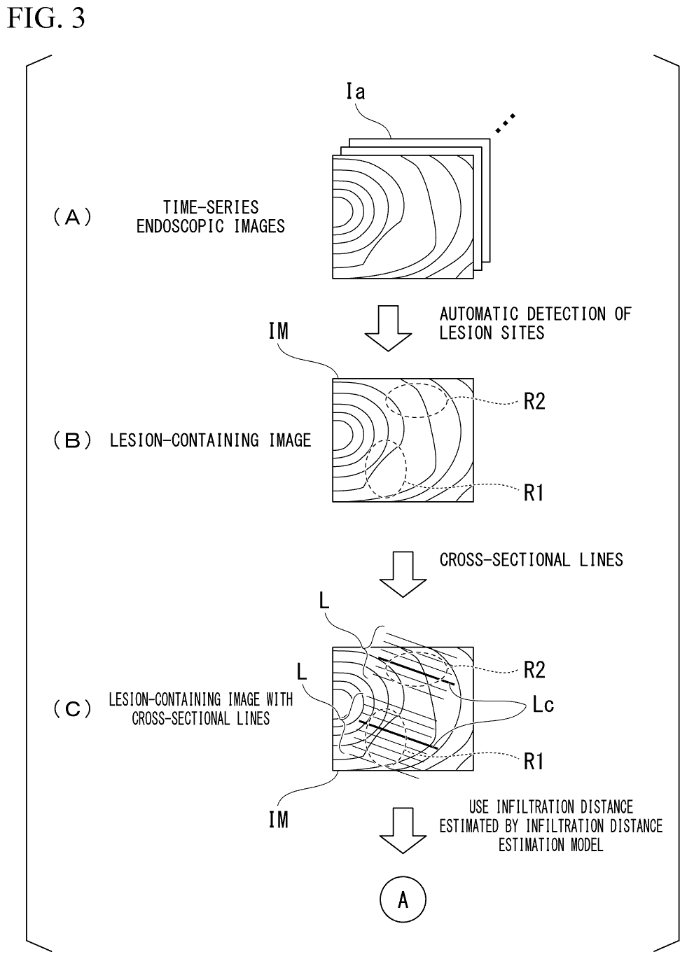

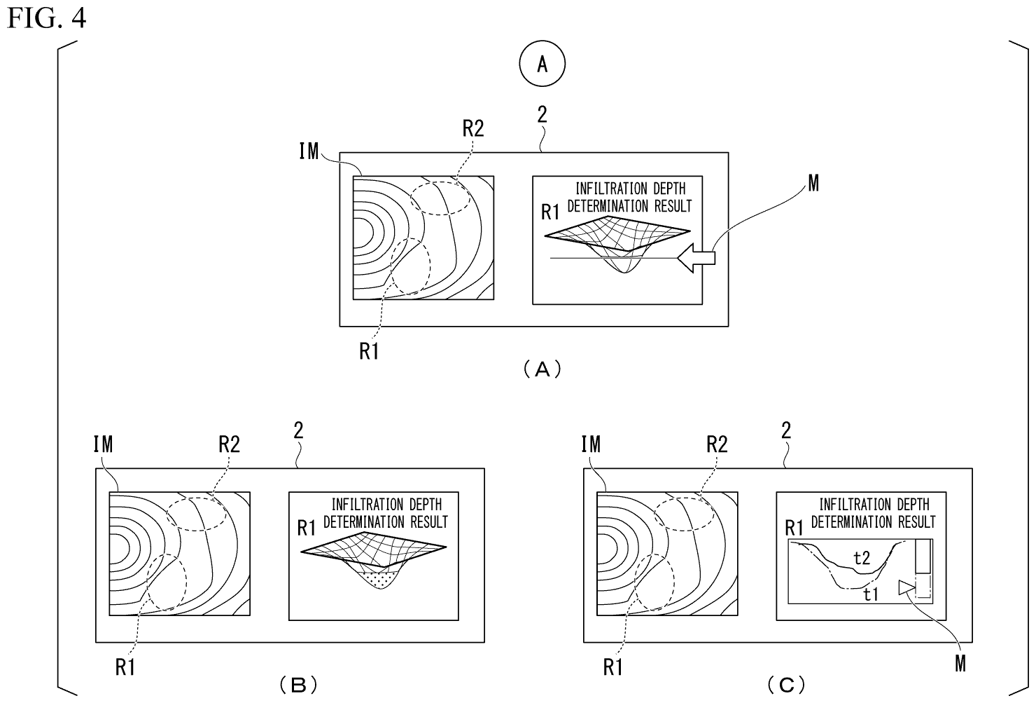

FIG. 3 is a first diagram for describing the display processing flow based on the infiltration distance of a lesion site, performed by the image processing device 1 according to some example embodiments of the present disclosure. FIG. 4 is a second diagram for describing the display processing flow based on the infiltration distance of a lesion site, performed by the image processing device 1 according to some example embodiments of the present disclosure.

First, as shown in (A) of FIG. 3, the image processing device 1 acquires time-series endoscopic images Ia from the endoscope 3. Then, as shown in (B) of FIG. 3, the image processing device 1 recognizes, among the acquired endoscopic images Ia, those corresponding to lesion-containing images either through automatic detection of the lesion site by the lesion detection model or through designation using the still-image save button on the operation unit 36. Then, the image processing device 1 causes the display device 2 to display the recognized lesion containing image (that is, an image containing regions R that includes the lesion site). It should be noted that the image shown in (B) of FIG. 3 is an example in which there are two lesion sites, and therefore regions R1, R2 are shown as regions R that includes the lesion sites.

In a case where the image processing device 1 displays the lesion-containing image on the display device 2, it sets multiple cross-sections for regions R corresponding to each of the lesion sites, each of which includes an entire lesion site (for example, in a case where two lesion sites exist, a region R1 including one entire lesion site and a region R2 including the other entire lesion site, respectively), as shown in (C) of FIG. 3. For example, the image processing device 1 sets multiple cross-sections corresponding to the multiple cross-sectional lines L by setting multiple cross-sectional lines L parallel to the cross-sectional line “Lc” at minute equal intervals for each of the regions R1, R2 each including one entire lesion site.

Then, as shown in FIG. 4, the image processing device 1 causes the display device 2 to display the infiltration state relative to a predetermined threshold value based on the infiltration distance for each position in the lesion-containing image (that is, for each position along the cross-sectional line L) estimated using the infiltration distance estimation model. For example, as shown in (A) of FIG. 4, the image processing device 1 may display on the display device 2 an image of a three-dimensional model (hereinafter, may be referred to as “lesion 3D model”) representing the three-dimensional shape of the lesion site estimated based on the estimated infiltration distance, together with a mark M indicating a predetermined threshold value that is one of the criteria for determining the applicability of endoscopic resection to the lesion site. The image of the three-dimensional model (lesion 3D model) is represented by a three-dimensional shape using Computer Graphics (CG). Examples of the mark M include an arrow, a line, and a triangle. In the example shown in (A) of FIG. 4, the mark M is an arrow. The lesion 3D model may be presented using a graphical representation commonly used in Computer-Aided Design (CAD), such as a wireframe display.

Furthermore, for example, as shown in (B) of FIG. 4, the image processing device 1 may display an image of the estimated lesion 3D model on the display device 2, where a portion exceeding a predetermined threshold value is rendered in a different manner (for example, in a different color) from other portions, based on the estimated infiltration distance.

Moreover, for example, as shown in (C) of FIG. 4, the image processing device 1 may display on the display device 2 the infiltration distance along the cross-sectional line L that at least passes through the point of maximum infiltration distance based on the peak value of the infiltration distance estimated at time t2, together with the mark M, as well as the infiltration distance along the cross-sectional line L that at least passes through the point of maximum infiltration distance based on the peak value of the infiltration distance estimated at time t1, which is earlier than time t2. In the example shown in (C) of FIG. 4, the mark Mis a triangle. It should be noted that since the shape of the captured area may change in real time, the image processing device 1 also displays the infiltration distance estimated at a slightly earlier time together with the currently estimated infiltration distance. The time difference between time t1 and time t2 is, for example, 0.5 seconds. In the case of multiple lesions where infiltration distances are displayed in real time, each lesion image is stored in association with its respective lesion and the corresponding estimated infiltration distance. Then, if the images match (that is, the previous lesion image and the current lesion image are compared and are determined as having the similarity above a threshold value), the infiltration distance estimated from the current lesion image is displayed.

### (Functional Blocks of Image Processing Device)

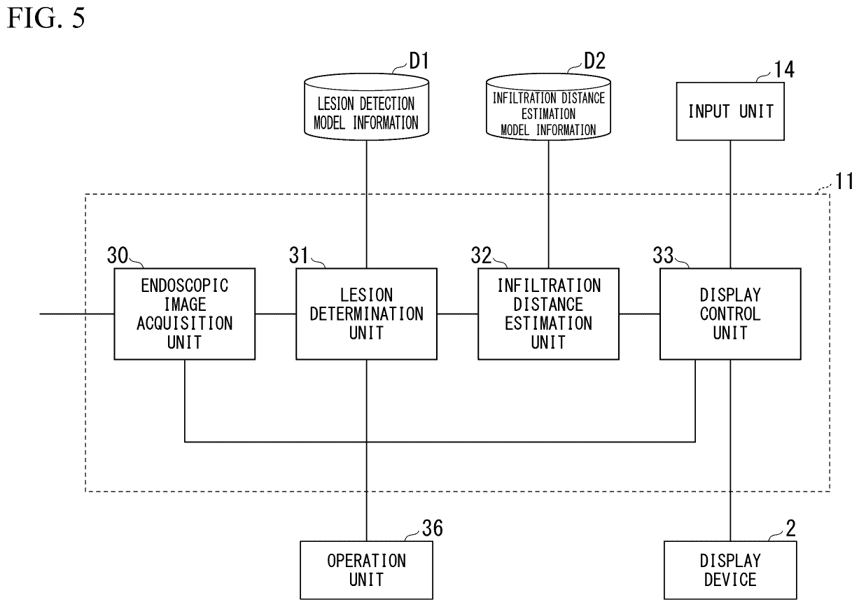

FIG. 5 is a diagram showing an example of the functional blocks of the image processing device 1 according to some example embodiments of the present disclosure. The processor 11 of the image processing device 1 has an endoscopic image acquisition unit 30, a lesion determination unit 31, an infiltration distance estimation unit 32, and a display control unit 33, as shown in FIG. 5. In FIG. 5, blocks that exchange data are connected by solid lines. However, the combination of blocks between which data is exchanged is not limited to this example.

The endoscopic image acquisition unit 30 acquires the endoscopic image Ia captured by the endoscope 3 at predetermined intervals via the interface 13. Then, the endoscopic image acquisition unit 30 supplies the acquired endoscopic image Ia to the lesion determination unit 31 and the display control unit 33, respectively.

The lesion determination unit 31 detects at least one lesion contained in the endoscopic image obtained during the endoscopic examination. For example, the lesion determination unit 31 determines whether or not the endoscopic image Ia supplied from the endoscopic image acquisition unit 30 is a lesion-containing image. In such a case, the lesion determination unit 31 detects an endoscopic image Ia that is a lesion-containing image, based on, for example, at least either a user input (that is, an external input) or an analysis result of the endoscopic image Ia. Then, if the lesion determination unit 31 detects an endoscopic image Ia that is a lesion containing image, the lesion determination unit 31 supplies the detected lesion containing image to the infiltration distance estimation unit 32. The lesion determination unit 31 is an example of the lesion detection means.

The following describes the detection of a lesion containing image based on user input. In such a case, in a case where the lesion determination unit 31 detects the still-image save button as being selected based on a signal supplied from the operation unit 36, it detects the endoscopic image Ia displayed on the display device 2 at the time of selection as a lesion-containing image. In such a case, the lesion determination unit 31 may detect the latest endoscopic image Ia supplied from the endoscopic image acquisition unit 30 at the time the still-image save button is selected as a lesion-containing image.

Next, the following describes the detection of a lesion-containing image based on the analysis results of the endoscopic image Ia. The lesion determination unit 31 inputs the endoscopic image Ia supplied from the endoscopic image acquisition unit 30 to a lesion detection model configured with reference to the lesion detection model information D1. Then, the lesion determination unit 31 determines whether or not the input endoscopic image Ia is a lesion-containing image, based on the information output by the lesion detection model in a case where the endoscopic image Ia is input. For example, the lesion detection model outputs a classification result regarding the presence or absence of a lesion site in the input endoscopic image Ia. Then, the lesion determination unit 31 determines whether or not the input endoscopic image Ia is a lesion-containing image based on the classification result.

The infiltration distance estimation unit 32 estimates the infiltration state of a lesion detected from an endoscopic image obtained during an endoscopic examination, and determines whether the infiltration state exceeds a predetermined threshold value. For example, the infiltration distance estimation unit 32 estimates the infiltration distance of a lesion site of the subject shown in the lesion-containing image supplied from the lesion determination unit 31, based on an infiltration distance estimation model configured by referring to the infiltration distance estimation model information D2. Then, the infiltration distance estimation unit 32 supplies the estimation result to the display control unit 33. In such a case, in a first example, the infiltration distance estimation unit 32 generates partial images by regularly dividing the lesion-containing image (for example, in a grid pattern), and inputs each divided partial image into the infiltration distance estimation model in sequence, thereby outputting the infiltration distance for each partial image output in sequence by the infiltration distance estimation model, to the display control unit 33. In a second example, the infiltration distance estimation unit 32 inputs the lesion-containing image into the infiltration distance estimation model, and outputs to the display control unit 33 an image showing the infiltration distance for each pixel of the lesion containing image, which is output by the infiltration distance estimation model. The infiltration distance estimation unit 32 is an example of the infiltration determination means.

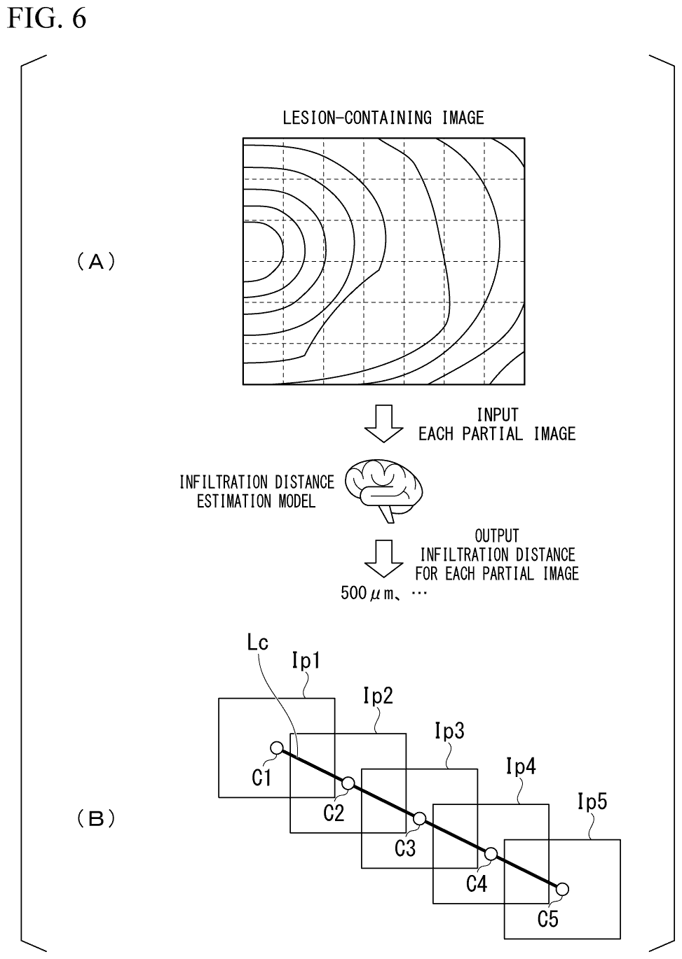

FIG. 6 is a diagram for describing the processing performed by the infiltration distance estimation unit 32 according to some example embodiments of the present disclosure. (A) of FIG. 6 shows an overview of the infiltration distance estimation process performed by the infiltration distance estimation unit 32 based on the first example mentioned above. In the example overview of the infiltration distance estimation process shown in (A) of FIG. 6, the infiltration distance estimation unit 32 generates a total of 42 partial images for each lesion containing image by dividing the lesion-containing image into 7 parts horizontally and 6 parts vertically in a grid pattern, and inputs each partial image into the lesion detection model to acquire the infiltration distance at the center position of each partial image. As a result, the infiltration distance estimation unit 32 acquires a distribution (map) of infiltration distances on the lesion-containing image, which is necessary for generating a map of the infiltration distance of the subject corresponding to the endoscopic image Ia (hereinafter, may be referred to as “infiltration distance map”). The infiltration distance map may be a heat map in which the darker the color, the longer the infiltration distance of the lesion site. It should be noted that instead of dividing a lesion containing image into a grid pattern, the infiltration distance estimation unit 32 may generate partial images by allowing overlap between the partial images and making the distance between the center positions of adjacent partial images shorter than the length of the partial images. This makes it possible to obtain a more detailed distribution of the infiltration distance on the lesion-containing image.

Moreover, in a case where the infiltration distance estimation unit 32 is set not to generate and display an infiltration distance map, the infiltration distance estimation unit 32 may estimate the infiltration distance by limiting it to positions along the specified cross-sectional line Lc, and acquire the infiltration distance required for the lesion cross-sectional view. (B) of FIG. 6 is a diagram showing an overview of a method for estimating the infiltration distance along the cross-sectional line Lc. In the example of (B) of FIG. 6, the infiltration distance estimation unit 32 sets points C1 through C5 at equal intervals on the cross-sectional line Lc, and sets partial images Ip1 through Ip5 which are square regions centered on the points C1 through C5, respectively. Then, the infiltration distance estimation unit 32 inputs the partial images Ip1 through Ip5 in sequence to the infiltration distance estimation model, and acquires the infiltration distances output in sequence by the infiltration distance estimation model as the infiltration distances at the points C1 through C5. It should be noted that the infiltration distance estimation unit 32 may acquire the infiltration distance for each of the cross-sectional lines L parallel to the cross-sectional line Lc by using the infiltration distance estimation model in the same manner as for the cross-sectional line Lc.

In addition, the infiltration distance estimation unit 32 or the display control unit 33 may interpolate the infiltration distance output by the infiltration distance estimation model using any interpolation process, and further calculate a function representing the infiltration distance at any point on the cross-sectional line Lc. This allows the infiltration distance estimation unit 32 or the display control unit 33 to accurately identify the shape of the lesion site in a case where the cross-sectional line Lc and the cross-sectional line L are taken as cross-sections. As a result, the display control unit 33 can display on the display device 2 a lesion 3D model that represents the smooth shape of the lesion site. Moreover, in a case where generating an infiltration distance map, the infiltration distance estimation unit 32 or the display control unit 33 may similarly generate an infiltration distance map by interpolating the infiltration distance output by the infiltration distance estimation model in the vertical and horizontal directions using any interpolation process.

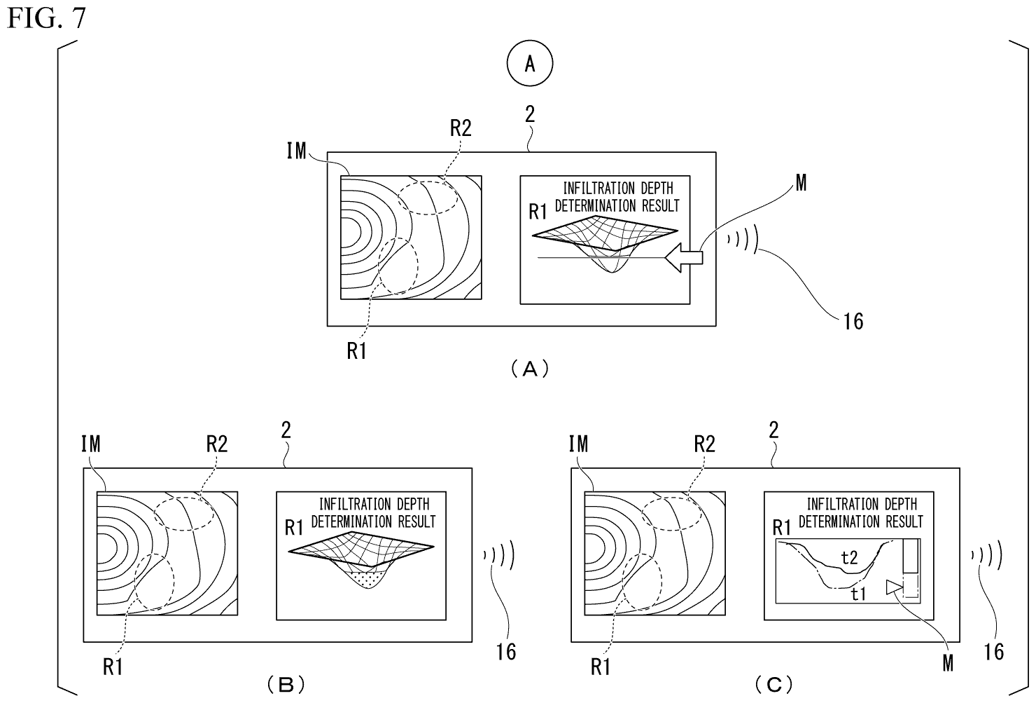

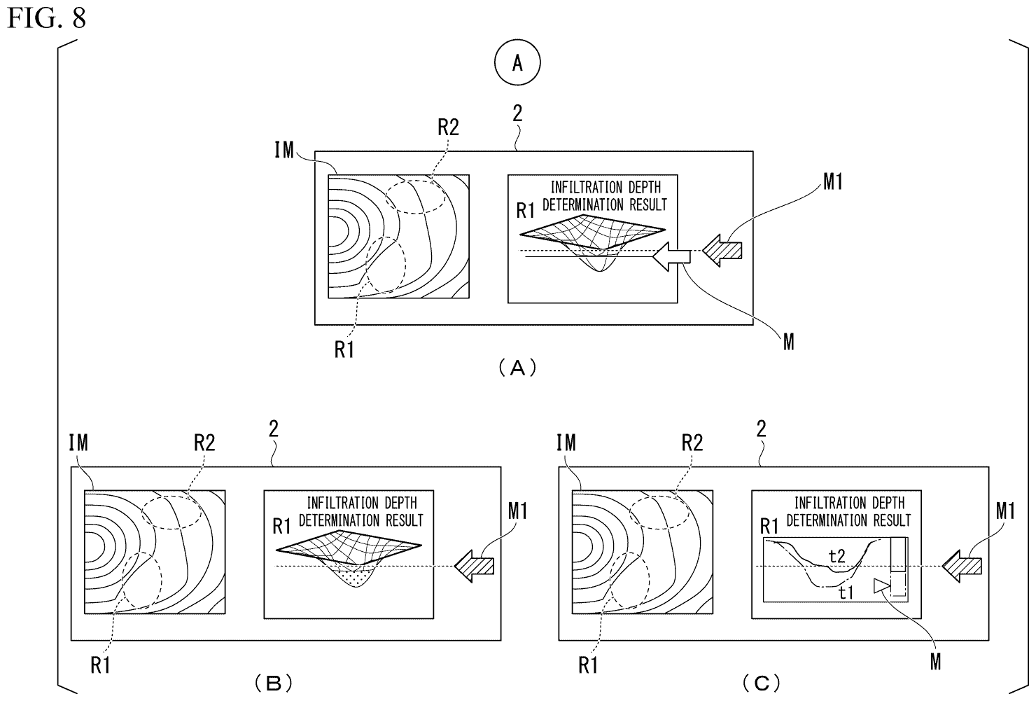

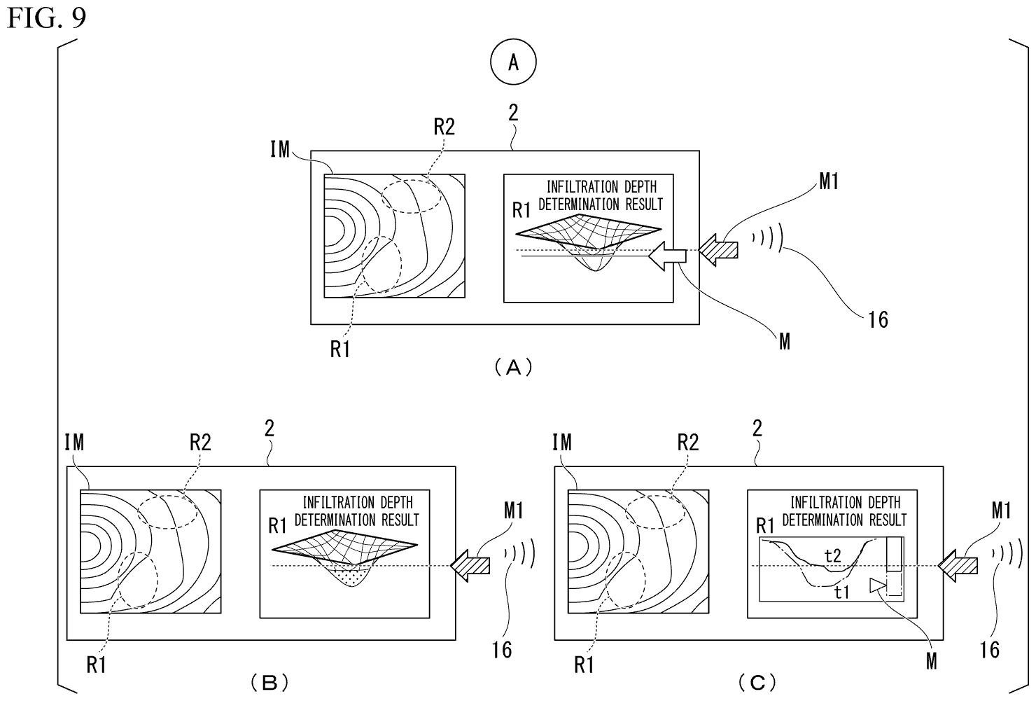

Referring again to FIG. 5, the display control unit 33 will be described. If the infiltration state is determined as exceeding a predetermined threshold value, the display control unit 33 generates an alert for the user conducting the endoscopic examination. FIG. 7 is a first diagram for describing an alert generated by the display control unit 33 according to some example embodiments of the present disclosure. FIG. 8 is a second diagram for describing an alert generated by the display control unit 33 according to some example embodiments of the present disclosure. FIG. 9 is a third diagram for describing an alert generated by the display control unit 33 according to some example embodiments of the present disclosure.

For example, in a case where the infiltration distance estimation unit 32 determines the infiltration state as exceeding a predetermined threshold value, the display control unit 33 may cause the display device 2 to display a screen similar to the screen shown in (A), (B), or (C) of FIG. 4, as shown in (A) of FIG. 7, indicating that the infiltration state exceeds the predetermined threshold value (that is, there is a possibility of additional surgical resection), and may cause the sound output unit 16 to output a warning sound or voice guidance to notify the user. The warning sound or voice guidance output from the sound output unit 16 may have a different sound pattern (melody) or pitch depending on the infiltration state (for example, the longest infiltration distance).

Moreover, for example, if the infiltration distance estimation unit 32 determines the infiltration state as exceeding a predetermined threshold value, the display control unit 33 may notify the user that the infiltration state exceeds the predetermined threshold value by displaying on the display device 2 a screen indicating the threshold value, using an arrow or a triangle as the mark M shown in (A) of FIG. 4 or (C) of FIG. 4. Also, for example, if the infiltration distance estimation unit 32 determines the infiltration state as exceeding a predetermined threshold value, the display control unit 33 may notify the user that the infiltration state exceeds the predetermined threshold value by displaying on the display device 2 a screen indicating the lesion site where the threshold value is exceeded in a different color from other areas, as shown in (B) of FIG. 4. Moreover, for example, if the infiltration distance estimation unit 32 determines the infiltration state as exceeding a predetermined threshold value, the display control unit 33 may notify the user that the infiltration state exceeds the predetermined threshold value by displaying on the display device 2 a screen including a meter indicating the infiltration distance at each time and a mark M indicating the threshold value, as shown in (C) of FIG. 4.

It should be noted that the display control unit 33 may cause the display device 2 to display a mark M1 indicating the maximum infiltration distance of the lesion in the past together with the portions (A), (B), or (C) of FIG. 4, as shown in FIG. 8. Moreover, the display control unit 33 may cause the display device 2 to display a mark M1 indicating the maximum infiltration distance of the lesion in the past together with the portions (A), (B), or (C) of FIG. 7, as shown in FIG. 9.

Furthermore, the display control unit 33 may cause the display device 2 to display another screen. For example, in a case where the lesion determination unit 31 detects a lesion-containing image, the display control unit 33 may cause the display device 2 to display the latest lesion-containing image in addition to or instead of the latest endoscopic image Ia. Also, for example, the display control unit 33 may cause the display device 2 to display an infiltration distance map. In the case of displaying an infiltration distance map, the image processing device 1 may generate an infiltration distance map for the entire lesion-containing image or an infiltration distance map for a part of the lesion-containing image and cause the display device 2 to display it. It should be noted that in a case where the image processing device 1 generates an infiltration distance map for a part of a lesion-containing image and displays it on the display device 2, it can display on the display device 2 an infiltration distance map that is limited to the area of interest to the examiner.

The display control unit 33 may cause the display device 2 to also display a lesion cross-sectional view. Moreover, the display control unit 33 may cause the display device 2 to display, on the lesion cross-sectional view, the mucosal layer (M), the muscularis mucosae (MM), and the submucosa (SM) that constitute the wall layer of the large intestine, which is the subject of the examination.

A specific example of a method for generating a lesion cross-sectional view will be described below. In a first example, the display control unit 33 generates a lesion cross-sectional view based on the estimated depth (width) of each wall layer of the large intestine output by an infiltration distance estimation model that has been trained to estimate the depth of each wall layer of the large intestine in addition to the infiltration distance in a case where a partial image or a lesion cross-sectional view is input. In such a case, the display control unit 33 may interpolate the estimated results of the depth of each wall layer along the cross-sectional line Lc for each wall layer, and generate a lesion cross-sectional view based on the depth of each wall layer obtained by the interpolation. In a second example, in the case where information indicating the standard value of the depth of each wall layer of the large intestine is stored in the memory 12, the display control unit 33 makes reference to this information and generates a cross-sectional view of the lesion in which the depth of each wall layer is set to the standard value mentioned above. It should be noted that in the lesion cross-sectional view, for example, the bottom of the submucosal layer (SM) is illustrated to as to align with the lower edge of the diagram. The display control unit 33 is an example of the display control means.

Each of the endoscopic image acquisition unit 30, the lesion determination unit 31, the infiltration distance estimation unit 32, and the display control unit 33 can be implemented by, for example, the processor 11 executing a program. Moreover, the necessary program may be preliminarily recorded on any non-volatile storage medium and installed as needed to implement each of the endoscopic image acquisition unit 30, the lesion determination unit 31, the infiltration distance estimation unit 32, and the display control unit 33. At least some of the endoscopic image acquisition unit 30, the lesion determination unit 31, the infiltration distance estimation unit 32, and the display control unit 33 are not limited to being implemented by a software program. For example, each of the endoscopic image acquisition unit 30, the lesion determination unit 31, the infiltration distance estimation unit 32, and the display control unit 33 may be implemented by any combination of hardware, firmware, and software. Moreover, at least some of the endoscopic image acquisition unit 30, the lesion determination unit 31, the infiltration distance estimation unit 32, and the display control unit 33 may be implemented using a user-programmable integrated circuit, such as a Field-Programmable Gate Array (FPGA) or a microcontroller. In such a case, the endoscopic image acquisition unit 30, the lesion determination unit 31, the infiltration distance estimation unit 32, and the display control unit 33 described above are implemented using a program that causes this integrated circuit to configure the endoscopic image acquisition unit 30, the lesion determination unit 31, the infiltration distance estimation unit 32, and the display control unit 33 described above. Moreover, at least some of the endoscopic image acquisition unit 30, the lesion determination unit 31, the infiltration distance estimation unit 32, and the display control unit 33 may be composed of an Application Specific Standard Produce (ASSP), an Application Specific Integrated Circuit (ASIC), or a quantum processor (quantum computer control chip). Thus, the endoscopic image acquisition unit 30, the lesion determination unit 31, the infiltration distance estimation unit 32, and the display control unit 33 may each be implemented by various types of hardware. The above also applies to other example embodiments of the present disclosure. Furthermore, each of the endoscopic image acquisition unit 30, the lesion determination unit 31, the infiltration distance estimation unit 32, and the display control unit 33 may be implemented using cloud computing technology or similar means, through cooperation among multiple computers.

### (Processing Performed by Endoscopic Examination System)

The above processing performed by the endoscopic examination system 100 is an example, and the processing performed by the endoscopic examination system 100 according to an example embodiment of the present disclosure is not limited to the processing described above. For example, the endoscopic examination system 100 may perform the processing described below.

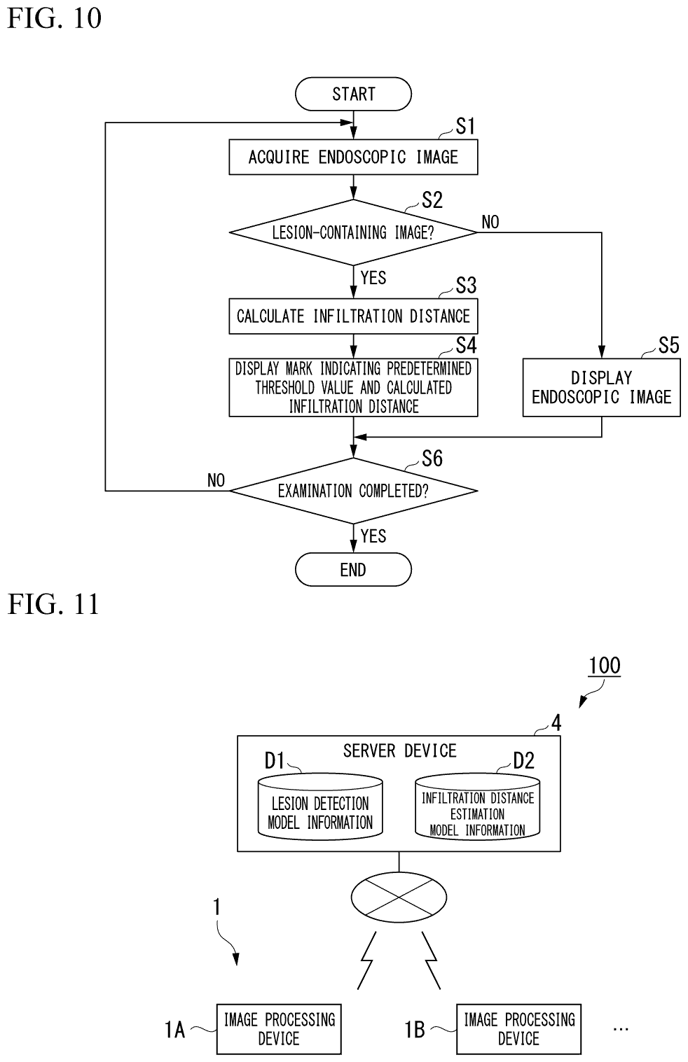

FIG. 10 is a diagram showing a processing flow example of the endoscopic examination system 100 according to some example embodiments of the present disclosure. Next, the processing of displaying the infiltration state relative to a predetermined threshold value in a visually discernible manner on the display device 2 based on the infiltration distance estimated by the image processing device 1 of the endoscopic examination system 100 will be described, with reference to FIG. 10.

The image processing device 1 acquires an endoscopic image Ia (Step S1). For example, the endoscopic image acquisition unit 30 of the image processing device 1 receives the endoscopic image Ia from the endoscope 3 via the interface 13.

The image processing device 1 determines whether or not the acquired endoscopic image Ia corresponds to a lesion containing image that includes a lesion site (Step S2). For example, the image processing device 1 makes the determination mentioned above based on information output by the lesion detection model configured based on the lesion detection model information D1 in a case where an endoscopic image Ia is input to the lesion detection model.

Then, if the endoscopic image Ia acquired in Step S1 is determined as being a lesion-containing image (YES in Step S2), the image processing device 1 calculates the infiltration distance (Step S3). In such a case, the image processing device 1 acquires the infiltration distance output by the infiltration distance estimation model in a case where the lesion-containing image or a partial image thereof is input to the infiltration distance estimation model configured based on the infiltration distance estimation model information D2. Then, the image processing device 1 displays the infiltration state relative to a predetermined threshold value in a visually discernible manner. For example, the image processing device 1 causes the display device 2 to display a mark M indicating a predetermined threshold value and the calculated infiltration distance (Step S4). In such a case, the image based on the infiltration distance is, for example, an image displayed on the display device 2 in a manner that allows the infiltration state relative to the threshold value to be visually discernible as shown in any one of FIG. 4 or FIG. 7 through FIG. 9.

Moreover, if the endoscopic image Ia acquired in Step S1 is determined as not being a lesion-containing image (NO in Step S2), the image processing device 1 causes the display device 2 to display the endoscopic image Ia acquired in Step S1 (Step S5).

After Step S4 or Step S5, the image processing device 1 determines whether or not the endoscopic examination has completed (Step S6). For example, if a predetermined input to the input unit 14 or the operation unit 36 is detected, the image processing device 1 determines the endoscopic examination as being completed. Then, if the endoscopic examination is determined as being completed (YES in Step S6), the image processing device ends the processing.

Moreover, if the endoscopic examination is determined as not being completed (NO in Step S6), the image processing device 1 returns the processing to Step S1. The image processing device 1 then executes the processes of Step S1 through Step S5 on the endoscopic image Ia newly generated by the endoscope 3.

### (Advantages)

The endoscopic examination system 100 according to an example embodiment of the present disclosure has been described in the foregoing. In the image processing device 1 (an example of the endoscopic examination assistance device) of the endoscopic examination system 100, the lesion determination unit 31 (an example of the lesion detection means) detects at least one lesion present in an endoscope image acquired during an endoscopic examination. The infiltration distance estimation unit 32 (an example of the infiltration determination means) estimates the infiltration state of the lesion detected from the endoscopic image and determines if the infiltration state exceeds a predetermined threshold value. The display control unit 33 (an example of the display control means) displays the infiltration state relative to the threshold value in a visually discernible manner. This image processing device 1 assists the examiner, such as a doctor, in determining the necessity of endoscopic resection of a lesion site. In other words, the image processing device 1 can optimize endoscopic resection of a lesion site and facilitate minimally invasive medical procedures. As a result, it is possible to assist the examiner, such as doctor.

### Modifications of Example Embodiment

Next, preferred modifications of the example embodiment described above will be described. The following modifications may be applied in combination to the example embodiment described above.

### Modification 1

The cross-sectional lines Lc and the cross-sectional lines L may be specified by the user. For example, in a case where the lesion determination unit 31 detects a lesion-containing image, the display control unit 33 accepts a user input specifying a cross-sectional line Lc on the lesion-containing image or the latest endoscopic image Ia. For example, the image processing device 1 causes the display device 2 to display the screen shown in (B) of FIG. 3 as well as a message such as “Specify cross-sectional line Lc” that prompts the user to specify the cross-sectional line Lc. In the display, the user may specify the cross-sectional line Lc, for example, by using a mouse or, if the display device 2 includes a touch panel, by utilizing the touch panel functionality. The display control unit 33 generates a lesion cross-sectional view in which the cross-sectional line Lc specified by the user input is taken as a cross-section. The display control unit 33 may then cause the display device 2 to display the generated lesion cross-sectional view. In such a case, the display control unit 33 may receive a designation of the cross-sectional line Lc after displaying the infiltration distance map. As a result, the examiner can perform the operation of specifying the cross-sectional line Lc while verifying the lesion's location using the infiltration distance map. As a result, it is possible to suitably assist the specification of the cross-sectional line Lc performed by the examiner. The display control unit 33 may receive a user input specifying a cross-sectional line Lc on the infiltration distance map, and generate a lesion cross-sectional view in which the cross-sectional line Lc specified by the user input is used as a cross-section. The display control unit 33 may then cause the display device 2 to display the generated lesion cross-sectional view.

### Modification 2

The image processing device 1 may process a video image composed of the endoscopic images Ia generated during an endoscopic examination after the examination. For example, the image processing device 1 may sequentially perform the processing shown in the processing flow of FIG. 10 on the time-series endoscopic images Ia that constitute the video image, in a case where the video image to be processed is specified based on a user input via the input unit 14 or similar means at any point in time following the examination. Subsequently, if the target video image is determined as having ended in Step S6, the image processing device 1 may terminate the processing shown in the processing flow, and if the target video image has not ended, it may return the processing to Step S1 and perform the processing shown in the processing flow on the subsequent endoscopic image Ia in the time series as the target image. This allows the examiner to obtain various information related to the lesion, which facilitates a more informed judgment of the lesion.

### Modification 3

The lesion detection model information D1 and the infiltration distance estimation model information D2 may be stored in a storage device separate from the image processing device 1. FIG. 11 is a schematic configuration diagram of an endoscopic examination system 100 according to some example embodiments of the present disclosure. It should be noted that, for simplicity, components such as the display device 2 and the endoscope 3 are omitted from FIG. 11. The endoscopic examination system 100 includes a server device 4 that stores lesion detection model information D1 and infiltration distance estimation model information D2. Moreover, the endoscopic examination system 100 also includes multiple image processing devices 1 (1A, 1B, . . . ) capable of data communication with the server device 4 via a network. In such a case, each image processing device 1 makes reference to the lesion detection model information D1 and the infiltration distance estimation model information D2 via the network. The interface 13 of each image processing device 1 includes a communication interface such as a network adapter for performing communication. In this endoscopic examination system 100, each image processing device 1 can suitably execute processing related to lesion detection by making reference to the lesion detection model information D1 and the infiltration distance estimation model information D2, similarly to the example embodiment described above.

### Second Example Embodiment

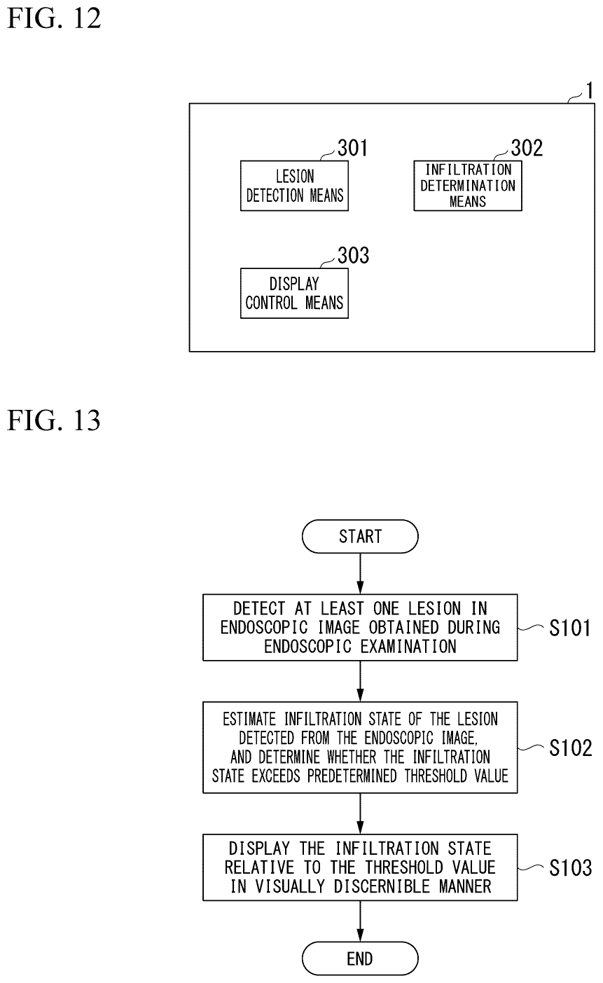

FIG. 12 is a diagram showing a configuration example of an image processing device 1 according to some example embodiments of the present disclosure. The image processing device 1 is an example of the endoscopic examination assistance device. As shown in FIG. 12, the image processing device 1 includes a lesion detection means 301, an infiltration determination means 302, and a display control means 303. The lesion detection means 301 detects at least one lesion contained in an endoscopic image obtained during an endoscopic examination. The infiltration determination means 302 estimates the infiltration state of the lesion detected from the endoscopic image and determines if the infiltration state exceeds a predetermined threshold value. The display control means 303 displays the infiltration state relative to the threshold value in a visually discernible manner.

The lesion detection means 301 can be implemented using, for example, the functions of the lesion determination unit 31 exemplified in FIG. 5. The infiltration determination means 302 can be implemented using, for example, the functions of the infiltration distance estimation unit 32 exemplified in FIG. 5. The display control means 303 can be implemented using, for example, the functions of the display control unit 33 exemplified in FIG. 5.

FIG. 13 is a diagram showing a processing flow example of the image processing device 1 according to some example embodiments of the present disclosure. The lesion detection means 301 detects at least one lesion contained in an endoscopic image obtained during an endoscopic examination (Step S101). The infiltration determination means 302 estimates the infiltration state of the lesion detected from the endoscopic image and determines if the infiltration state exceeds a predetermined threshold value (Step S102). The display control means 303 displays the infiltration state relative to the threshold value in a visually discernible manner (Step S103).

### (Advantages)

The examiner such as doctor can be assisted by this image processing device 1.

It should be noted that, in the example embodiments of the present disclosure (including modifications), the order of processing steps may be altered, provided that appropriate processing is ensured.

In each of the example embodiments described above, the program can be stored using various types of non-transitory computer-readable media and can be supplied to a processor or the like, which is a computer. Non-transitory computer-readable media include various types of tangible storage media. Examples of non-transitory computer-readable media include magnetic storage media (for example, floppy disks, magnetic tapes, hard disk drives), magneto optical storage media (for example, magneto-optical disks), CD-ROMs (Read-Only Memory), CD-Rs, CD-R/Ws, and semiconductor memories (for example, mask ROMs, PROMs (Programmable ROMs), EPROMs (Erasable PROMs), flash ROMs, and RAMs (Random Access Memory). Moreover, the program may also be provided to the computer by various types of transitory computer-readable media. Examples of transitory computer-readable media include electrical signals, optical signals, and electromagnetic waves. The transitory computer-readable medium can supply the program to the computer via a wired communication path such as an electric wire or an optical fiber, or via a wireless communication path.

Several example embodiments of the present disclosure have been described, however, these example embodiments are merely examples and do not limit the scope of the disclosure. Various additions, omissions, substitutions, and modifications may be made to these example embodiments without departing from the scope of the disclosure.

The whole or part of the example embodiments (including modifications) disclosed above can be described as, but not limited to, the following supplementary notes.

According to each example aspect of the present disclosure, it is possible to assist an examiner such as a doctor.

While preferred example embodiments of the disclosure have been described and illustrated above, it should be understood that these are exemplary of the disclosure and are not to be considered as limiting. Additions, omissions, substitutions, and other modifications can be made without departing from the scope of the present disclosure. Accordingly, the disclosure is not to be considered as being limited by the foregoing description, and is only limited by the scope of the appended claims.

### (Supplementary Note 1)

An endoscopic examination assistance device comprising:
a lesion detection means that detects at least one lesion contained in an endoscopic image obtained during an endoscopic examination;
an infiltration determination means that estimates an infiltration state of the lesion detected from the endoscopic image and determines whether the infiltration state exceeds a predetermined threshold value; and
a display control means that displays the infiltration state relative to the threshold value in a visually discernible manner.

### (Supplementary Note 2)

The endoscopic examination assistance device according to supplementary note 1, comprising
an alert generation means that generates an alert directed to an examiner conducting the endoscopic examination in a case where it is determined that the infiltration state exceeds the threshold value.

### (Supplementary Note 3)

The endoscopic examination assistance device according to supplementary note 2, wherein:
the alert generated by the alert generation means is a warning sound or a voice guidance; and
comprising
a notification means that outputs the warning sound or the voice guidance.

### (Supplementary Note 4)

The endoscopic examination assistance device according to supplementary note 3, wherein
the notification means
changes a sound pattern or sound pitch based on the infiltration state and outputs the warning sound or the voice guidance.

### (Supplementary Note 5)

The endoscopic examination assistance device according to supplementary note 2, wherein:
the alert generated by the alert generation means is a mark indicating a predetermined threshold value; and
comprising
a display control means that displays the mark.

### (Supplementary Note 6)

The endoscopic examination assistance device according to supplementary note 2, wherein:
the alert generated by the alert generation means refers to a lesion site exceeding a predetermined threshold value, which is displayed in a color different from other areas; and
comprising
a display control means that displays a lesion site exceeding the predetermined threshold value.

### (Supplementary Note 7)

The endoscopic examination assistance device according to supplementary note 2, wherein:
the alert generated by the alert generation means is a screen including a meter showing an infiltration distance at each time and a mark showing a threshold value; and
comprising
a display control means that displays the screen.

### (Supplementary Note 8)

The endoscopic examination assistance device according to any one of supplementary notes 1 through 7, wherein
the display control means
displays a maximum value of an infiltration distance of the lesion in the past.

### (Supplementary Note 9)

The endoscopic examination assistance device according to any one of supplementary notes 1 through 8, wherein
the predetermined threshold value is
a predetermined threshold value that serves as a criterion for determining the applicability of endoscopic resection to a lesion site.

### (Supplementary Note 10)

The endoscopic examination assistance device according to any one of supplementary notes 1 through 9, wherein
at least one of the lesion detection means and the infiltration determination means
uses a machine-learned trained model.

### (Supplementary Note 11)

An endoscopic examination system comprising:
the endoscopic examination assistance device according to any one of claims 1 through 10; and
a display device that displays a screen under the control of the endoscopic examination assistance device.

### (Supplementary Note 12)

A processing method comprising:
detecting at least one lesion contained in an endoscopic image obtained during an endoscopic examination;
estimating an infiltration state of the lesion detected from the endoscopic image and determining whether the infiltration state exceeds a predetermined threshold value; and
displaying the infiltration state relative to the threshold value in a visually discernible manner.

### (Supplementary Note 13)

The processing method according to supplementary note 12, comprising
generating an alert directed to an examiner conducting the endoscopic examination in a case where it is determined that the infiltration state exceeds the threshold value.

### (Supplementary Note 14)

The processing method according to supplementary note 13, wherein:
the alert generated is a warning sound or a voice guidance; and
comprising
outputting the warning sound or the voice guidance.

### (Supplementary Note 15)

The processing method according to supplementary note 13, comprising
changing a sound pattern or sound pitch based on the infiltration state and outputting the warning sound or the voice guidance.

### (Supplementary Note 16)

The processing method according to supplementary note 13, wherein:
the alert generated is a mark indicating a predetermined threshold value; and
comprising
displaying the mark.

### (Supplementary Note 17)

The processing method according to supplementary note 13, wherein:
the alert generated refers to a lesion site exceeding a predetermined threshold value, which is displayed in a color different from other areas; and
comprising
displaying a lesion site exceeding the predetermined threshold value.

### (Supplementary Note 18)

The processing method according to supplementary note 13, wherein:
the alert generated is a screen including a meter showing an infiltration distance at each time and a mark showing a threshold value; and
comprising
displaying the screen.

### (Supplementary Note 19)

The processing method according to any one of supplementary notes 12 through 18, comprising
displaying a maximum value of an infiltration distance of the lesion in the past.

### (Supplementary Note 20)

The processing method according to any one of supplementary notes 12 through 19, wherein
the predetermined threshold value is
a predetermined threshold value that serves as a criterion for determining the applicability of endoscopic resection to a lesion site.

### (Supplementary Note 21)

The processing method according to any one of supplementary notes 12 through 20, comprising
executing, using a machine-learned trained model, steps of:
detecting at least one lesion contained in an endoscopic image obtained during the endoscopic examination; or estimating an infiltration state of the lesion detected from the endoscopic image and determining whether the infiltration state exceeds a predetermined threshold value.

### (Supplementary Note 22)

A program causing
a computer to execute steps of:
detecting at least one lesion contained in an endoscopic image obtained during an endoscopic examination;
estimating an infiltration state of the lesion detected from the endoscopic image and determining whether the infiltration state exceeds a predetermined threshold value; and
displaying the infiltration state relative to the threshold value in a visually discernible manner.

### (Supplementary Note 23)

The program according to supplementary note 22 that causes the computer to execute a step of
generating an alert directed to an examiner conducting the endoscopic examination in a case where it is determined that the infiltration state exceeds the threshold value.

### (Supplementary Note 24)

The program according to supplementary note 23, wherein:
the alert generated is a warning sound or a voice guidance; and
that causes the computer to execute a step of
outputting the warning sound or the voice guidance.

### (Supplementary Note 25)

The program according to supplementary note 24 that causes the computer to execute a step of
changing a sound pattern or sound pitch based on the infiltration state and outputting the warning sound or the voice guidance.

### (Supplementary Note 26)

The program according to supplementary note 23, wherein:
the alert generated is a mark indicating a predetermined threshold value; and
that causes the computer to execute a step of
displaying the mark.

### (Supplementary Note 27)

The program according to supplementary note 23, wherein:
the alert generated refers to a lesion site exceeding a predetermined threshold value, which is displayed in a color different from other areas; and
that causes the computer to execute a step of
displaying a lesion site exceeding the predetermined threshold value.

### (Supplementary Note 28)

The program according to supplementary note 23, wherein:
the alert generated is a screen including a meter showing an infiltration distance at each time and a mark showing a threshold value; and
that causes the computer to execute a step of
displaying the screen.

### (Supplementary Note 29)

The program according to any one of supplementary notes 22 through 28 that causes the computer to execute a step of
displaying a maximum value of an infiltration distance of the lesion in the past.

### (Supplementary Note 30)

The program according to any one of supplementary notes 22 through 29, wherein
the predetermined threshold value is
a predetermined threshold value that serves as a criterion for determining the applicability of endoscopic resection to a lesion site.

### (Supplementary Note 31)

The endoscopic examination assistance device according to any one of supplementary notes 22 through 30, that causes the computer, using a machine-learned trained model, to execute steps of:
detecting at least one lesion contained in an endoscopic image obtained during the endoscopic examination; or
estimating an infiltration state of the lesion detected from the endoscopic image and determining whether the infiltration state exceeds a predetermined threshold value.

What is claimed is:

1. An endoscopic examination assistance device comprising:
at least one memory configured to store instructions; and
at least one processor configured to execute the instructions to:
detect at least one lesion included in an endoscopic image obtained during an endoscopic examination;
estimate an infiltration state of the lesion detected from the endoscopic image;
determines whether or not an infiltration distance indicating the infiltration state exceeds a predetermined threshold value; and
display the infiltration distance relative to the threshold value in a visually discernible manner.

2. The endoscopic examination assistance device according to claim 1, wherein the at least one processor is configured to execute the instructions to generate an alert directed to an examiner conducting the endoscopic examination in a case where it is determined that the infiltration distance exceeds the threshold value.

3. The endoscopic examination assistance device according to claim 2, wherein the generated alert is a warning sound or a voice guidance, and
wherein the at least one processor is configured to execute the instructions to output the warning sound or the voice guidance.

4. The endoscopic examination assistance device according to claim 3, wherein the at least one processor is configured to:
change a sound pattern or sound pitch based on the infiltration state; and
output the warning sound or the voice guidance.

5. The endoscopic examination assistance device according to claim 2, wherein the generated alert is a mark indicating the predetermined threshold value, and
wherein the at least one processor is configured to execute the instructions to display the mark.

6. The endoscopic examination assistance device according to claim 2, wherein the generated alert indicates that the infiltration distance exceeds the predetermined threshold value, and
wherein the at least one processor is configured to execute the instructions to display a lesion site of the lesion in a different color from other sites.

7. The endoscopic examination assistance device according to claim 2, wherein the generated alert is a screen including a meter showing an infiltration distance at each time and a mark showing a threshold value, and
wherein the at least one processor is configured to execute the instructions to display the screen.

8. The endoscopic examination assistance device according to claim 1, wherein the at least one processor is configured to execute the instructions to display a maximum value of the infiltration distance of the lesion in the past.

9. The endoscopic examination assistance device according to claim 1, wherein the predetermined threshold value is a threshold value that serves as a criterion for determining the applicability of endoscopic resection to a lesion site.

10. The endoscopic examination assistance device according to claim 1, wherein at least one of detecting or determining are executed based on a machine-learned trained model.

11. An endoscopic examination system comprising:
the endoscopic examination assistance device according to claim 1; and
a display configured to display a screen under the control of the endoscopic examination assistance device.

12. A processing method comprising:
detecting at least one lesion contained in an endoscopic image obtained during an endoscopic examination;
estimating an infiltration state of the lesion detected from the endoscopic image;
determining whether or not an infiltration distance indicating the infiltration state exceeds a predetermined threshold value; and
displaying the infiltration distance relative to the threshold value in a visually discernible manner.

13. The processing method according to claim 12, further comprising generating an alert directed to an examiner conducting the endoscopic examination in a case where it is determined that the infiltration distance exceeds the threshold value.

14. The processing method according to claim 13, wherein the generated alert is a warning sound or a voice guidance, and
wherein the method further comprises outputting the warning sound or the voice guidance.

15. The processing method according to claim 14, further comprising:
changing a sound pattern or sound pitch based on the infiltration state; and
outputting the warning sound or the voice guidance.

16. The processing method according to claim 13, wherein the generated alert is a mark indicating the predetermined threshold value, and
wherein the method further comprises displaying the mark.

17. The processing method according to claim 13, wherein the generated alert indicates that the infiltration distance exceeds the predetermined threshold value, and
wherein the method further comprises displaying a lesion site of the lesion in a different color from other sites.

18. The processing method according to claim 13, wherein the generated alert is a screen including a meter showing an infiltration distance at each time and a mark showing a threshold value, and
wherein the method further comprises displaying the screen.

19. The processing method according to claim 12, further comprising displaying a maximum value of the infiltration distance of the lesion in the past.

20. A non-transitory storage medium storing a program causing a computer to execute:
detecting at least one lesion contained in an endoscopic image obtained during an endoscopic examination;
estimating an infiltration state of the lesion detected from the endoscopic image;
determining whether or not an infiltration distance indicating the infiltration state exceeds a predetermined threshold value; and
displaying the infiltration distance relative to the threshold value in a visually discernible manner.
